(19) United States
(12) Patent Application Publication
(10) Pub. No.: US 2011/0053191 A1
(43) Pub. Date: Mar. 3, 2011

(54) METHOD FOR RISK REDUCTION IN GLYCEMIC CONTROL

(76) Inventors: Georg Hess, Mainz (DE); Andrea Horsch, Mannheim (DE); Dietmar Zdunek, Tutzing (DE)

(21) Appl. No.: 12/938,430

(22) Filed: Nov. 3, 2010

Related U.S. Application Data
(63) Continuation of application No. PCT/EP2009/056388, filed on May 26, 2009.

(30) Foreign Application Priority Data
May 27, 2008 (EP) 08156982.4

(51) Int. Cl. G01N 33/68; C12M 1/34

(52) U.S. Cl. 435/7.92; 435/287.1

(57) ABSTRACT

Disclosed is a method for identifying a subject being susceptible to a therapy for intensive glycemic control, the subject suffering from diabetes and being in need for a therapy for intensive glycemic control, based on determining the amount of PLGF (placental growth factor) in a sample of the subject and comparing the thus determined amount to a reference amount. In a preferred embodiment, the method further includes determining at least one further marker selected from the group consisting of a cardiac troponin and a natriuretic peptide and comparing the determined amount(s) to a reference amount (amounts). Moreover, disclosed is a method for predicting the risk of an acute cardiovascular event in a subject who suffers from diabetes and is on intensive glycemic control. Further disclosed is a kit and a device adapted to carry out the method of the present invention.

### RELATED APPLICATIONS

This application is a continuation of PCT/EP2009/056388 filed May 26, 2009 and claims priority to EP 08156982.4 filed May 27, 2008.

### FIELD OF THE INVENTION

The present invention relates to a method for identifying a subject being susceptible to a therapy for intensive glycemic control, the subject suffering from diabetes and being in need for a therapy for intensive glycemic control, based on determining the amount of placental growth factor (PLGF) a sample of the subject and comparing the thus determined amount to a reference amount. In a preferred embodiment, the method further comprises determining at least one further marker selected from the group consisting of a cardiac troponin and a natriuretic peptide and comparing the determined amount(s) to a reference amount (amounts). Moreover, the present invention relates to a method for predicting the risk of an acute cardiovascular event in a subject who suffers from diabetes and is on intensive glycemic control. Further envisaged by the present invention is a kit and a device adapted to carry out the method of the present invention.

### BACKGROUND OF THE INVENTION

An aim of modem medicine is to provide personalized or individualized treatment regimens. Those are treatment regimens which take into account a patient's individual needs or risks.

Diabetes mellitus is characterized by disordered metabolism and hyperglycemia resulting from decreased levels of the hormone insulin with or without abnormal resistance to the effects of insulin. There are three major forms of diabetes mellitus: type 1, type 2, and gestational diabetes. Type 1 diabetes (frequently also referred to a juvenile diabetes) is usually caused by destruction of the pancreatic beta cells which produce insulin. Diabetes mellitus type 2, frequently also referred to as adult-onset diabetes or Type 2 diabetes, is a metabolic disorder that is primarily characterized by insulin resistance, hyperglycemia, and relative insulin deficiency. The prevalence of diabetes mellitus type 2 is rapidly increasing throughout the developed world, and there is evidence that this pattern will be followed in many other countries in coming years. Gestational diabetes is similar to type 2 diabetes since also insulin resistance is involved. Here, pregnancy related hormones can cause insulin resistance in individuals which are genetically predisposed to developing this condition.

Diabetes is linked with various comorbidities. Diabetes patients frequently suffer from diabetic retinopathy, diabetic nephropathy, diabetic neuropathy, peripherally vascular disease, high cholesterol levels, hypertension, atherosclerosis, renal failure and coronary artery disease. Particularly, patients with type 2 diabetes mellitus die of cardiovascular disease (CVD, particularly acute coronary events) at rates that are up to four times higher than for non-diabetic individuals of similar demographic characteristics

It has been believed for decades, that lowering the blood sugar levels to normal sugar levels would reduce the risk of the aforementioned diseases and particularly the risk of dying from CVD.

However, recently the ACCORD trial (Action to Control Cardiovascular Risk in Diabetes trial) was partly halted due to unforeseeable problems. The ACCORD trial is a research program that was designed to determine the best way to reduce the risk of myocardial infarction in individuals. In one part of the ACCORD study, it was analysed whether a tight glycemic control, i.e., a therapeutic strategy that lowers to blood sugar levels to nearly normal levels, would reduce the number of acute cardiovascular events in diabetes patients who are at high risk for having a cardiovascular disease event because of existing clinical or subclinical CVD or CVD risk factors. Tight glycemic control was achieved by a therapeutic strategy that targets the HbA1c level to levels of <6.0%. Unexpectedly, there were more deaths in the group with an intensive glycemic control than in the group of individuals whose blood sugar levels were less rigidly controlled. Therefore, the ACCORD investigators stopped the aforementioned study and recommended that the participants with an intensive glycemic control regimen should be put on a less intense regimen.

However, lowering the blood sugar to normal levels still would be beneficial in terms of the other comorbidities of diabetes. E.g, lowered blood sugar can protect against retinopathy, kidney disease and amputations. But, since the benefits of a tight glycemic control in patients with a cardiovascular disease are outweighed by the increased mortality, methods are required to identify those patients which are susceptible to a therapy for a tight glycemic control, and, thus, to identify those patients which would benefit from such a therapy without being at increased risk of a cardiovascular event.

Therefore, there is a need for measures which allow a reliable identification of diabetes patients susceptible to a therapy for intensive glycemic control.

The technical problem underlying the present invention can be seen as the provision of means and methods for complying with the aforementioned needs. The technical problem is solved by the embodiments characterized in the claims and herein below.

### SUMMARY OF THE INVENTION

Accordingly, the present invention relates to a method for identifying a subject being susceptible to a therapy for intensive glycemic control, the subject suffering from diabetes mellitus and, preferably, being in need for a therapy for intensive glycemic control, comprising the steps
a) determining the amount of PLGF (placental growth factor) in a sample of the subject,
b) comparing the amount of PLGF as determined in step a) to a suitable reference amount, and
c) identifying the subject.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of a subject in need of a therapy for an intensive glycemic control. The method may be carried out manually or assisted by automation. Preferably, step (a), (b) and/or (c) may in total or in part be assisted by automation, e.g., by a suitable robotic and sensory equipment for the determination in step (a) or a computer-implemented comparison in step (b).

### DETAILED DESCRIPTION OF THE INVENTION

The term “identifying” as used herein means determining whether a subject will be susceptible for a therapy for an intensive glycemic control or not. A subject who is susceptible to the therapy will benefit from the therapy and will not be at increased risk of acute cardiovascular events due to the therapy, whereas subject who is not susceptible to the therapy, preferably, will be at increased risk of acute cardiovascular events due to the intensive glycemic control. Therefore, it preferably should be avoided that a subject who is not susceptible to a therapy for intensive glycemic control is put an intensive glycemic control. The subject, however, will preferably benefit from a therapy for moderate glycemic control (the term “moderate glycemic control” is specified elsewhere herein).

It will be understood by those skilled in the art, such an assessment (whether a subject is susceptible to a therapy or not) is usually not intended to be correct for all (i.e., 100%) of the subjects to be identified. The term, however, requires that a statistically significant portion of subjects can be identified (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc.. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 40%, at least 50%, at least 60%, at least 70%, at least 80% or at least 90% of the subjects of a population can be properly identified by the method of the present invention.

The term “subject” as used herein relates to animals, preferably mammals, and, more preferably, humans.

However, it is envisaged in accordance with the aforementioned method of the present invention that the subject shall be “in need of a therapy for an intensive glycemic control”. Therefore, the subject, preferably suffers suffer from diabetes mellitus.

Diabetes mellitus (the terms “diabetes” and “diabetes mellitus” are used interchangeably herein) according to the present invention relates to all forms of diabetes mellitus, including type 1, type 2 and gestational diabetes. Preferably, diabetes relates to type 1 or type 2 diabetes and, more preferably, to type 2 diabetes.

Definitions of diabetes mellitus are known to the person skilled in the art and diagnostic criteria have been established by the World Health Organization (WHO) in 1985 and 1999, as well as by the American Diabetes Association (ADA) in 1997. Any patient fulfilling the criteria according to one or more of these definitions is considered a diabetes patient. Preferably, the diabetes patient is defined according to the WHO 1999 criteria.

Type 1 diabetes is also known as juvenile diabetes or insulin-dependent diabetes mellitus (IDDM).

Type 1 diabetes can be caused immunologically (subtype A) and or it can be idiopathic (subtype B). It is known in the art that type 1 diabetes is characterized by loss of the insulin-producing beta cells of the islets of Langerhans in the pancreas.

Type 2 diabetes is also known as adult-onset diabetes or non-insulin-dependent diabetes mellitus (NIDDM). Type 2 diabetes can either be accompanied by adipositas (type 2a) or not be accompanied by adipositas (type 2b). Type 2 diabetes is the most common prevalent form of diabetes and is found in over 90% of the diabetic patient population. Subjects suffering from type 2 retain a certain level of endogenous insulin secretory function. However, insulin levels are low relative to the magnitude of insulin resistance and glucose levels. Type 2 diabetes, preferably, can be assessed by determining the fasting blood glucose level. A fasting blood glucose or serum glucose concentration greater than 125 mg/dL (6.94 mmol/L), preferably, indicates diabetes type 2. Moreover, type 2 diabetes can be assessed by carrying out the glucose tolerance test which is well known in the art. Preferably, a blood sugar level of 200 mg of glucose or larger per dL of plasma two hours after the intake of 75 g glucose (after over-night fasting) indicates type 2 diabetes. In a glucose tolerance test 75 g of glucose are administered orally to the patient being tested after 10-12 hours of fasting and the blood sugar level is recorded immediately before taking the glucose and 1 and 2 hours after taking it. How to determine blood glucose is well known in the art.

Prior to carrying out the method of the present invention (or more precisely prior to obtaining the sample to be analyzed by the method of the present invention), the subject, preferably, shall have an HbA1c level (a definition for this level can be found elsewhere herein) between 7.5% and 11%, more preferably between 7.5% and 9%, and most preferably between 7.5% and 8.5%. The subject may take already some drugs that reduce the blood sugar level and, thus, the HbA1c level (a definition for the HbA1c level can be found elsewhere herein.

Moreover, the subject preferably shall also suffer from coronary artery disease or shall be at risk of suffering from coronary artery disease. The term “coronary artery disease”, abbreviated CAD, frequently also called coronary heart disease (CHD) or atherosclerotic heart disease, is well known in the art. Preferably, the term refers to a condition in which the blood vessels that supply blood and oxygen to the heart are narrowed. Coronary artery disease is usually caused by a condition called atherosclerosis, which occurs when fatty material and a plaque builds up on the walls of your arteries. This causes them to get narrow. Particularly, CAD is the result of the accumulation of atheromatous plaques within the walls of the arteries that supply the myocardium (the muscle of the heart). Preferably, a subject with stable CAD has at least 50% stenosis (and, 50% thus occlusion), in at least one major coronary artery. How to assess the degree of occlusion of a coronary artery is well known in the art, preferably, the degree is assessed by coronary angiography. While the symptoms and signs of coronary artery disease are noted in the advanced state of disease, most individuals with coronary artery disease show no evidence of disease for decades as the disease progresses before the first onset of symptoms of an acute event, often a “sudden” heart attack, finally arise.

Thus, as used herein, the term “coronary artery disease”, preferably, shall include stenosis, atherosclerosis of the coronary vessels or occlusions. Preferably, the term coronary artery disease refers to stable coronary artery disease. Stable coronary artery disease, preferably, does not include acute cardiovascular syndromes. Particularly, stable coronary artery disease does not include STEMI (ST-elevation myocardial infarction); NSTEMI (non ST-elevation myocardial infarction) and unstable angina pectoris (but is does include stable angina pectoris). However, the subject shall may have a history of events belonging to the acute cardiovascular syndrome, i.e., the subject may have exhibited at least one acute cardiovascular event in the past (but not recently, particularly not within a month, three months and, more preferably, not within one year prior to carry out the method of the present invention (or, more precisely, prior to obtaining the sample to be analyzed by the method of the present invention). Acute cardiovascular events are, preferably, acute coronary syndromes (ACS). ACS patients can show unstable angina pectoris (UAP) or myocardial infarction (MI). MI can be an ST-elevation MI (STEMI) or a non-ST-elevated MI (NSTEMI). The occurring of an ACS can be followed by a left ventricular dysfunction (LVD) and symptoms of heart failure. How to diagnose an acute cardiovascular event is well known in the art.

A subject who at risk of suffering from coronary artery disease, preferably, is a subject for which at least two of the following criteria apply: cigarette smoking, obesity (body mass index larger than 30 kg/m2 and, more preferably, larger than 32 kg/m2), arterial hypertension (untreated systolic blood pressure >140 mm Hg or diastolic blood pressure >95 mm Hg, or on medication for lowering blood pressure), hyperlipidemia (untreated LDL-C>130 mg/dl (3.38 mmol/1), or on medication for lowering lipids), and low HDL-C (high density lipoprotein C<40 mg/dl (1.04 mmol/l) for men and <50 mg/dl (1.29 mmol/l) for women).

The term “therapy for an intensive glycemic control” encompasses those treatment regimens that aim to significantly decrease the blood glucose level in a subject as mentioned above. As used herein, the term “therapy for an intensive glycemic control”, preferably, relates a treatment regimen that is capable of significantly reducing the fasting, prandial and/or postprandial blood glucose levels (preferably, blood serum glucose levels) and, more preferably, the glycosylated hemoglobin (HbA1c) level.

It is to understood that a reduction of the level of blood glucose does not refer to the reduction of the level blood glucose in a host taken at a particular point in time, since blood glucose levels can vary throughout the day, e.g., due to food intake. Rather, the reduction of the level of blood glucose, preferably, relates to a reduction of the blood glucose level, preferably of the average blood glucose level, in a subject over a period of time. Preferably, the period of time is more than one month, two months, three months, more preferably more than six months and, more preferably, more than one year, and even more preferably, more than 3 years and, most preferably, more than 10 years. The reduction of blood glucose in a subject suffering from diabetes may be assessed by determining the area under a glycemic control curve that is formed by plotting, e.g., the minute-to-minute changes in blood glucose levels in a subject over a given time period. Preferably, an intensive glycemic control is achieved when the blood glucose levels of a subject suffering from diabetes are the same or nearly the same as the blood glucose levels of subjects/a subject not suffering from diabetes over a given period of time, and, thus, when the area under the glycemic control curve is the same or nearly the same as the area under the corresponding curve of a subject not suffering from diabetes.

It is known in the art that the HbA1c level (glycated/glycosylated hemoglobin level) is proportional to average blood glucose concentration over the previous four weeks to three months. Therefore, a therapy for an intensive glycemic control is a treatment regimen that reduces the HbA1c level to certain levels, preferably, to levels that are the same or nearly the same of subjects not suffering from diabetes. Preferably, the HbA1c level is indicated in %, indicating the HbA1c concentration as a percentage of total hemoglobin.

Generally, HbA1c levels found in subjects not suffering from diabetes are within a range of about 4% to 5.9%. Accordingly, a therapy for an intensive glycemic control is, preferably, a treatment regimen that reduces the HbA1c level to 6.5% of total hemoglobin or lower, to 6.0% or lower, to 5.5% or lower, or to 5.2 or lower. Particularly preferred HbA1c levels to be achieved by the therapy are 6.5% or lower, and 6.0% or lower. However, although it is contemplated that the therapy for intensive glycemic control significantly lowers the HbA1c/average blood glucose amounts, it is not contemplated to lower the HbA1c/average blood glucose amounts to amounts that are lower than the amounts of subjects that are apparently healthy with respect to diabetes. Accordingly, the therapy for intensive glycemic control shall, preferably, target the HbA1c amounts to a range of between 4.0% to 52%, 4.0% to 5.5%, 4.0% to 6.0%, 4.0% to 6.5%, and, more preferably, between a range of 4.5% to 5.2%, 4.5% to 5.5%, even more preferably, to a range between 4.5% to 6.0%, and, most preferably, to a range between 4.5% to 6.5% of total hemoglobin.

HbA1c is frequently also referred to as glycosylated hemoglobin or glycated hemoglobin or hemoglobin Alc, Hb1c. The term is well known in the art. HbA1c is the product of a non-enzymatic glycation of the hemoglobin B chain. It is known in the art that its production depends on the blood sugar level and the life of the erythrocytes, and that HbA1c reflects the average blood sugar levels of the preceding four to six weeks. It is known, that diabetes patients whose HbA1c value is well adjusted by intensive diabetes treatment (e.g lower than 6.5% of the total hemoglobin) are better protected against microangiopathy. The skilled person knows how to determine the HbA1c level.

How to reduce the HbA1c level and the average blood glucose in diabetes patients and, thus, how to carry out a therapy for intensive glycemic control is well known in the art. Preferably, the treatment is by the administration of drugs. Preferably, the drug is selected from the group consisting of sufonylurea, an alpha-glucosidase inhibitor, a biguanide, metformin, a meglitinide, a thiazolidinedione, and insulin. More preferably, the drug is insulin. Also contemplated are combinations of the aforementioned drugs. It is clear, that the drugs shall be administered on a regular basis in order to achieve an intensive glycemic control. E.g., it may be required to take more than 4 shots of insulin a day or using an insulin pump. Moreover, it is clear, that the HbA1c amounts and/or blood glucose amounts shall be frequently determined in order to monitor the effect on the blood sugar level. E.g., the blood sugar level may be measured more than seven times a day.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “PLGF” relates to the placental growth factor or to variants thereof. PLGF is a type of vascular endothelial growth factor known to be expressed not only in placental cells but also many nonplacental cells including endothelial cells which are involved in blood vessel formation. Human PLGF is a 149-amino-acid-long polypeptide and is highly homologous (53% identity) to the platelet-derived growth factor-like region of human vascular endothelial growth factor (VEGF). Like VEGF, PLGF has angiogenic activity in vitro and in vivo. For example, biochemical and functional characterization of PLGF derived from transfected COS-1 cells revealed that it is a glycosylated dimeric secreted protein capable of stimulating endothelial cell growth in vitro (Maqlione 1993, Oncogene 8(4):925-31). Preferably, PLGF refers to human PLGF (see, e.g., Genebank accession number P49763, GI: 17380553.

The term “variants” in this context of the present invention relates to peptides which are substantially similar to the peptides. The term “substantially similar” is well understood by the person skilled in the art. In particular, a variant may be an isoform or allele which shows amino acid exchanges compared to the amino acid sequence of the most prevalent peptide isoform in the human population. Moreover, it is to be understood that a variant as referred to in accordance with the present invention shall have an amino acid sequence which differs due to at least one amino acid substitution, deletion and/or addition wherein the amino acid sequence of the variant is still, preferably, at least 60%, 70%, 80%, 85%, 90%, 92%, 95%, 97%, 98%, or 99% identical with the amino sequence of PLGF. Substantially similar are also degradation products, e.g., proteolytic degradation products, which are still recognized by the diagnostic means or by ligands directed against the respective full-length peptide. The term “variant” in the present context is also meant to relate to splice variants. Known splice variants of PLGF are PLGF-1 (149 amino acids), PLGF-2 (170 amino acids) and PLGF-3 (221 amino acids) (see e.g., Cai, J., Ahmad, S., Jiang, W. G., Huang, J., et al. (2003). Activation of Vascular Endothelial Growth Factor Receptor-1 Sustains Angiogenesis and Bcl-2 Expression via the Phosphatidylinositol 3-Kinase Pathway in Endothelial Cells. Diabetes, vol. 52, pp.2959-2968).The term “variant” also relates to a post-translationally modified peptide such as glycosylated or phosphorylated peptide. A “variant” is also a peptide which has been modified after collection of the sample, for example by covalent or non-covalent attachment of a label, particularly a radioactive or fluorescent label, to the peptide.

The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used.

Determining the amount of PLGF or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e., a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. The means comprise immunoassay devices and methods which may utilize labeled molecules in various sandwich, competition, or other assay formats. The assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. The methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys analyzers), CBA (an enzymatic Cobalt Binding Assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprise the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, the method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable lable prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. The secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art (e.g., using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available e.g., from Molecular Probes (Oregon). Also the use of quantum dots as fluorescent labels is contemplated. Typical radioactive labels include 35S, 1251, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other dectection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing the ligand are well known and include, but are not limited to ionic, hydrophobic, covalent interactions and the like. It is also contemplated to use “suspension arrays” as arrays according to the present invention (Nolan 2002, Trends Biotechnol, 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e., automatically provide the desired assessment in a suitable output format. Based on the comparison of the amount determined in step a) and the reference amount, it is possible to assess whether a subject is susceptible for a therapy for intensive glycemic control, i.e., belonging to the group of subjects which can be successfully treated by an intensive glycemic control (and thus subjects which benefit from the therapy without the adverse side effects described herein, particularly of an acute cardiovascular event). Therefore, the reference amount is to be chosen so that either a difference or a similarity in the compared amounts allows identifying those test subjects which belong into the group of subjects susceptible for a therapy for an intensive glycemic control or identifying those test subjects which are not susceptible for a therapy for an intensive glycemic control.

Accordingly, the term “reference amount” as used herein refers to an amount which allows assessing whether a subject in need thereof will be susceptible for a therapy for an intensive glycemic control as referred to above. Accordingly, the reference may either be derived from (i) a subject known to have been successfully treated, i.e., without the occurrence of adverse effects, particularly of a cardiovascular event (particularly, a myocardial infarction), or (ii) a subject known to have been not successfully treated, i.e., a subject which developed an acute cardiovascular event (particularly, a myocardial infarction) or which died due to cardiovascular complications after and/or did not derive benefits from the treatment regimen. Moreover, the reference amount may define a threshold amount, whereby an amount of PLGF lower than the threshold shall be indicative for a subject being susceptible for a therapy for an intensive glycemic control (and, thus, is not at increased risk of cardiovascular events due to the therapy) while an amount larger than the threshold amount shall be an indicator for a subject which can not be treated successfully by an intensive glycemic control (and, thus, is indicative for a subject not being susceptible to the therapy since he would be at increased risk of cardiovascular events due to the therapy). The reference amount applicable for an individual subject may vary depending on various physiological parameters such as age, gender, or subpopulation, as well as on the means used for the determination of the polypeptide or peptide referred to herein. A suitable reference amount may be determined by the method of the present invention from a reference sample to be analyzed together, i.e., simultaneously or subsequently, with the test sample.

Accordingly, a reference amount defining a threshold amount for PLGF as referred to in accordance with the present invention is 25 pg/ml, more preferably, 20 pg/ml and, most preferably, 16 pg/ml.

Preferably, an amount of PLGF in a sample of a subject lower than the reference amount is indicative for a subject being susceptible to a therapy for intensive glycemic control.

Preferably, an amount of PLGF in a sample of a subject larger than the reference amount is indicative for a subject not being susceptible to a therapy for intensive glycemic control.

In the studies underlying the present invention, the amounts of PLGF, troponin T, and NT-proBNP were determined in a cohort of 891 subjects suffering from diabetes mellitus (See Examples). It was analyzed whether these markers correlate with cardiovascular events in a follow-up period of twelve years. The results showed that subjects with increased levels of PLGF are at elevated risk of suffering from a cardiovascular event, particularly, from an acute coronary syndrome. Also, subjects with increased amounts of troponin T and NT-proBNP are at elevated risk of suffering from the cardiovascular event.

The studies underlying the present invention strongly suggest that subjects which suffer from diabetes and which are in need of a therapy for an intensive glycemic control will not benefit from the therapy when having increased levels of PLGF. Subjects with increased levels of PLGF have a reduced blood flow. If those subjects are on a therapy for an intensive glycemic control, the blood sugar levels are significantly reduced. The present invention is based on the finding that, as a consequence of the significant reduction of the blood sugar level, fatal events occur more frequently in these individuals, if the amount of PLGF is increased. Therefore, those subjects with increased amounts of PLGF will not benefit from a therapy for an intensive glycemic control, and therefore are not susceptible to the therapy. However, they may be susceptible to a therapy for moderate glycemic control (see herein below).

Taken together, determining the amount of PLGF and comparing the, thus, determined amount of PLGF to a suitable reference amount is required to reliably identify those subjects which are susceptible or not susceptible to a therapy for an intensive glycemic control, i.e., for a good control of the blood sugar. An amount of PLGF in a sample of the subject lower than a suitable reference amount, preferably, indicates that a subject can be successfully treated by applying a therapy for an intensive control (i.e., without being at elevated risk of adverse side effects, particularly cardiovascular complications such as myocardial infarction), whereas an amount of the PLGF in a sample of the subject larger that a suitable reference amount, preferably, indicates that the subject is not susceptible to a therapy for an intensive glycemic control since that subject is at elevated risk of suffering from an adverse side effect of the therapy.

In a preferred embodiment of the method of the present invention also the amount of at least one further marker selected from the group consisting of a cardiac troponin and a natriuretic peptide is determined in a sample of the subject and compared to a suitable reference amount for the at least one further marker. Preferably, the further marker is a cardiac troponin, and, more preferably, troponin T. The at least further marker may be determined in the same sample for which the amount of PLGF is determined, or in a different sample.

The determination of at least one further marker allows that a statistically more significant portion of subjects can be correctly identified and, thus, adds further diagnostic and prognostic value. However, as described above the determination of an angiogenesis marker alone allows that a statistically significant portion of subjects can be correctly identified.

The term “cardiac troponin” refers to all troponin isoforms expressed in cells of the heart and, preferably, the subendocardial cells. These isoforms are well characterized in the art as described, e.g., in Anderson 1995, Circulation Research, vol. 76, no. 4: 681-686 and Ferrieres 1998, Clinical Chemistry, 44: 487-493. Preferably, cardiac troponin refers to troponin T and/or troponin I, and, most preferably, to troponin T. It is to be understood that isoforms of Troponins may be determined in the method of the present invention together, i.e., simultaneously or sequentially, or individually, i.e., without determining the other isoform at all. Amino acid sequences for human troponin T and human troponin I are disclosed in Anderson, loc cit and Ferrieres 1998, Clinical Chemistry, 44: 487-493.

The term “cardiac troponin” encompasses also variants of the aforementioned specific Troponins, i.e., preferably, of troponin T or troponin I. Such variants have at least the same essential biological and immunological properties as the specific cardiac Troponins. In particular, they share the same essential biological and immunological properties if they are detectable by the same specific assays referred to in this specification, e.g., by ELISA Assays using polyclonal or monoclonal antibodies specifically recognizing the cardiac Troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific troponin. Variants may be allelic variants or any other species specific homologs, paralogs, or orthologs. Moreover, the variants referred to herein include fragments of the specific cardiac Troponins or the aforementioned types of variants as long as these fragments have the essential immunological and biological properties as referred to above. Such fragments may be, e.g., degradation products of the Troponins. Further included are variants which differ due to posttranslational modifications such as phosphorylation or myristylation.

The term “natriuretic peptide” comprises atrial natriuretic peptide (ANP)-type and brain natriuretic peptide (BNP)-type peptides and variants thereof having the same predictive potential. Natriuretic peptides according to the present invention comprise ANP-type and BNP-type peptides and variants thereof (see e.g., Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP, and variants thereof. ANP and BNP are the active hormones and have a shorter half-life than their respective inactive counterparts, NT-proANP and NT-proBNP. BNP is metabolised in the blood, whereas NT-proBNP circulates in the blood as an intact molecule and as such is eliminated renally. The in-vivo half-life of NTproBNP is 120 min longer than that of BNP, which is 20 min (Smith 2000, J Endocrinol. 167: 239-46.). Preanalytics are more robust with NT-proBNP allowing easy transportation of the sample to a central laboratory (Mueller 2004, Clin Chem Lab Med 42: 942-4.). Blood samples can be stored at room temperature for several days or may be mailed or shipped without recovery loss. In contrast, storage of BNP for 48 hours at room temperature or at 4° Celsius leads to a concentration loss of at least 20% (Mueller loc.cit.; Wu 2004, Clin Chem 50: 867-73.). Therefore, depending on the time-course or properties of interest, either measurement of the active or the inactive forms of the natriuretic peptide can be advantageous.

The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc. cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein. The NT-proBNP referred to in accordance with the present invention further encompasses allelic and other variants of the specific sequence for human NT-proBNP discussed above. Specifically, envisaged are variant polypeptides which are on the amino acid level at least 60% identical, more preferably at least 70%, at least 80%, at least 90%, at least 95%, at least 98% or at least 99% identical, to human NT-proBNP. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human NT-proBNP as long as the polypeptides have NT-proBNP properties. NT-proBNP properties as referred to herein are immunological and/or biological properties. Preferably, the NT-proBNP variants have immunological properties (i.e., epitope composition) comparable to those of NT-proBNP. Thus, the variants shall be recognizable by the aforementioned means or ligands used for determination of the amount of the natriuretic peptides. Biological and/or immunological NT-proBNP properties can be detected by the assay described in Karl et al. (Karl 1999, Scand J Clin Invest 59:177-181), Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide.

Preferably, a reference amount defining a threshold amount for a cardiac troponin, particularly for troponin T, as referred to in accordance with the present invention is 30 pg/ml, more preferably, 20 pg/ml and, even more preferably, 10 pg/ml.

Preferably, an amount of a cardiac troponin, particularly of troponin T lower than the reference amount is, more preferably, indicative for a subject being susceptible to a therapy for intensive glycemic control (provided that the amount of the other markers referred to herein, if determined, also indicate that the subject is susceptible to the therapy, thus are also lower than the reference amount).

Preferably, an amount of a cardiac troponin, particularly of troponin T larger than the reference amount is, more preferably, indicative for a subject not being susceptible to a therapy for intensive glycemic control (provided that the amounts of the other markers referred to herein, if determined, also indicate that the subject is not susceptible to the therapy, thus are also larger than the reference amount).

Preferably, a reference amount defining a threshold for NT-proBNP as referred to in accordance with the present invention is, preferably, 300 pg/ml, more preferably, 250 pg/ml and, even more preferably, 200 pg/ml, and most preferably 150 pg/ml.

Preferably, an amount of a natriuretic peptide lower than the reference amount is indicative for a subject being susceptible to a therapy for intensive glycemic control (provided that the amount of the other markers referred to herein also indicate that the subject is susceptible to the therapy).

Preferably, an amount of a natriuretic peptide larger than the reference amount is indicative for a subject not being susceptible to a therapy for intensive glycemic control (provided that the amount of the other markers referred to herein also indicated that the subject is not susceptible to the therapy).

In the case that the amount of a natriuretic peptide and/or a cardiac troponin is determined in addition to PLGF, and the amounts of the various markers are contradicting (e.g., one amount lower than the reference amount, and one higher than the reference amount and vice versa), the subject needs to be carefully monitored if he receives a treatment for an intensive glycemic control. It is particularly contemplated that the amounts of the various biomarkers are being determined again after a certain period of time, e.g., after one month or six months.

A subject who is not susceptible to an intensive glycemic therapy (since it would put the subject at high risk of adverse side effects), preferably, is susceptible to a therapy for a moderate glycemic control. The therapy for moderate glycemic control targets the HbA1c amount to amounts to a range of between 6.5% and 8.0%, between 6.5% and 7.5%, more preferably between 7.0%and 7.5% and most preferably between 7.0% and 8.0% of total hemoglobin. How to target the HbA1c to the aforementioned ranges is well known in the art.

Accordingly, the present invention also relates to a method for determining whether a subject who suffers from diabetes is susceptible to an intensive glycemic control therapy or to a moderate glycemic control therapy, comprising the steps of
a) determining the amount of PLGF in a sample of the subject,
b) comparing the amount of PLGF as determined in step a) to a suitable reference amount, and
c) determining whether a subject is susceptible to the intensive or the moderate control therapy.

Preferably, an amount, in a sample of a subject, of PLGF larger than the reference amount indicates that the subject is susceptible to a therapy for moderate glycemic control, whereas an amount, in a sample of a subject, of PLGF lower than the reference amount indicates that the subject is susceptible to a therapy for intensive glycemic control. A subject who is susceptible to a therapy for intensive glycemic control, preferably, will derive maximal benefits from the therapy without being at increased risk of adverse side effects. He may, of course, also benefit from a therapy for moderate glycemic control, however, an intensive glycemic control is more beneficial. A subject with increased PLGF amounts (larger than the reference) will be at increased risk of cardiovascular events if being on intensive control, however, he still benefits from a therapy that aims to reduce the blood sugar level moderately.

It is also contemplated to determine the amounts of the other markers (a cardiac troponin and/or a natriuretic peptide) and to compare the amounts to reference amounts as described herein above (see above, the definitions and reference amounts apply mutatis mutandis).

Moreover, the present invention relates to a device adapted to carry out the method of the present invention. Particularly, the present invention relates to a device for identifying a subject being susceptible to a therapy for intensive glycemic control comprising
a) means for determining the amount of PLGF in a sample of a subject suffering from diabetes and, preferably, being in need of a therapy for intensive glycemic control, and
b) means for comparing the amount determined by the means to a reference amount, whereby a subject being susceptible therapy for intensive glycemic control is identified.

Preferably, the device also comprises means for determining the amount at least one further marker selected from the group consisting of a natriuretic peptide and a cardiac troponin and means for comparing the amount(s) as determined by the means to a suitable reference amount(s).

The term “device” as used herein relates to a system of means comprising at least the aforementioned means operatively linked to each other as to allow the prediction. Preferred means for determining the amount of PLGF and a cardiac Troponin, and a natriuretic peptide, and means for carrying out the comparison are disclosed above in connection with the method of the invention. How to link the means in an operating manner will depend on the type of means included into the device. For example, where means for automatically determining the amount of the peptides are applied, the data obtained by the automatically operating means can be processed by, e.g., a computer program in order to obtain the desired results. Preferably, the means are comprised by a single device in such a case. The device may accordingly include an analyzing unit for the measurement of the amount of the peptides or polypeptides in an applied sample and a computer unit for processing the resulting data for the evaluation. Alternatively, where means such as test stripes are used for determining the amount of the peptides or polypeptides, the means for comparison may comprise control stripes or tables allocating the determined amount to a reference amount. The test stripes are, preferably, coupled to a ligand which specifically binds to the peptides or polypeptides referred to herein. The strip or device, preferably, comprises means for detection of the binding of the peptides or polypeptides to the ligand. Preferred means for detection are disclosed in connection with embodiments relating to the method of the invention above. In such a case, the means are operatively linked in that the user of the system brings together the result of the determination of the amount and the prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the natriuretic peptide, Plasmon surface resonace devices, NMR spectrometers, mass-spectrometers etc.) or evaluation units/devices referred to above in accordance with the method of the invention.

Also envisaged by the present invention is a kit adapted to carry out the method of the present invention. Particularly, the present invention relates to a kit, the kit comprising instructions for carrying out the method, and
a) means for determining the amounts of PLGF in a sample of a subject suffering from diabetes and, preferably, being in need of a therapy for intensive glycemic control, and
b) means for comparing the amounts determined by the means to a reference amount, allowing identifying a subject being susceptible to a therapy for intensive glycemic control.

The term “kit” as used herein refers to a collection of the aforementioned means, preferably, provided in separately or within a single container. The container, also preferably, comprises instructions for carrying out the method of the present invention. In addition the kit, preferably, comprises means for determining the amount at least one further marker selected from the group consisting of a natriuretic peptide and a cardiac troponin and means for comparing the amount(s) as determined by the means to a suitable reference amount(s).

Moreover, the present invention relates to the use of PLGF for identifying a subject being susceptible to a therapy for intensive glycemic control. Also, the present invention envisages the use of PLGF and at least one further marker selected from the group consisting of a natriuretic peptide and a cardiac troponin for identifying a subject being susceptible to a therapy for intensive glycemic control.

The definitions and explanations of the terms given above apply mutatis mutandis for the preferred methods, the devices and kits referred to in the following.

The present invention also relates to a method for predicting the risk of an acute cardiovascular event in a subject who suffers from diabetes and who is on intensive glycemic control (and, thus, receives a therapy for intensive glycemic control), comprising the steps of
a) determining the amount of PLGF in a sample of the subject,
b) comparing the amount of PLGF as determined in step a) to a suitable reference amount, and
c) predicting the risk of an acute cardiovascular event in the subject.

In a preferred embodiment of the aforementioned method, at least one further marker selected from the group consisting of a cardiac troponin and a natriuretic peptide is determined.

Preferred reference amounts for the various amounts are given herein above.

The term “predicting” as used to assessing the probability according to which a subject who suffers from diabetes and is on intensive glycemic control (and, thus, has due to a therapy for intensive glycemic control HbA1c (and/or blood glucose) levels as indicated herein above) will develop a cardiovascular event, preferably an acute cardiovascular event within a defined time window (predictive window) in the future. The predictive window is an interval in which the subject will develop a cardiovascular event or will die according to the predicted probability. The predictive window may be the entire remaining lifespan of the subject upon analysis by the method of the present invention. Preferably, however, the predictive window is an interval of one month, six months or one, two, three, four, five or ten years after carrying out the method of the present invention (more preferably and precisely, after the sample to be analyzed by the method of the present invention has been obtained). As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc.. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “predicting the risk of an acute cardiovascular event” as used herein means that it the subject to be analyzed by the method of the present invention is allocated either into the group of subjects of a population having a normal, i.e., non-elevated and, thus, average risk for developing an acute cardiovascular event, or into a group of subjects having a elevated risk, or into a group of subjects having a significantly elevated risk. An elevated risk as referred to in accordance with the present invention also means that the risk of developing a cardiovascular event within a predetermined predictive window is elevated for a subject with respect to the average risk for a cardiovascular event in a population of subjects as defined herein. Preferably, for a predictive window of one year, the average risk is within the range of 2.0 and 3.0%, preferably, 2.5%. An elevated risk as used herein, preferably, relates to a risk of more than 3.0%, preferably, more than 4.0%, and, most preferably within 3.0% and 8.0%, with respect to a predictive window of one year. A significantly elevated risk as used herein, preferably relates to a risk more than 5.0%, preferably within the range of 5.0% and 8.0%, or even higher with respect to a predictive window of one year.

Acute cardiovascular events are, preferably, acute coronary syndromes (ACS). ACS patients can show unstable angina pectoris (UAP) or myocardial infarction (MI). MI can be an ST-elevation MI (STEMI) or a non-ST-elevated MI (NSTEMI). The occurring of an ACS can be followed by a left ventricular dysfunction (LVD) and symptoms of heart failure. How to diagnose an acute cardiovascular event is well known in the art.

Preferably, an amount of PLGF in a sample of a subject larger than the reference amount is indicative for a subject being at elevated risk of an acute cardiovascular event.

Preferably, an amount of PLGF in a sample of a subject lower than the reference amount is indicative for a subject not being at elevated risk, and, thus, being on average risk for an acute cardiovascular event.

If in addition to PLGF at least one further marker selected from the group consisting of a cardiac troponin and a natriuretic peptide is determined, the following applies:

Preferably, an amount of a cardiac troponin, particularly of troponin T lower than the reference amount is, more preferably, indicative for a subject not being at elevated risk (and thus for a subject being at average risk) of an acute cardiovascular event (provided that the amount of the other markers referred to herein also indicate the same).

Preferably, an amount of a cardiac troponin, particularly of troponin T larger than the reference amount is, more preferably, indicative for a subject being at elevated risk of an acute cardiovascular event (provided that the amount of the other markers referred to herein also indicate the same).

Preferably, an amount of a natriuretic peptide lower than the reference amount is indicative for a subject not being at elevated risk (and thus for a subject being at average risk) of an acute cardiovascular event (provided that the amount of the other markers referred to herein also indicate the same).

Preferably, an amount of a natriuretic peptide larger than the reference amount is indicative for a subject being at elevated risk of an acute cardiovascular event (provided that the amount of the other markers referred to herein also indicate the same).

Furthermore, the present invention concerns a device for predicting the risk of an acute cardiovascular event in a subject who suffers from diabetes an is on intensive glycemic control comprising
a) means for determining the amounts of PLGF in a sample of a subject who suffers from diabetes and is on intensive glycemic control, and
b) means for comparing the amounts determined by the means to a reference amount, allowing predicting the risk of an acute cardiovascular event in a subject who suffers from diabetes and is on intensive glycemic control.

Also envisaged by the present invention is a kit adapted to carry out the aforementioned method of the present invention, the kit comprising instructions for carrying out the method, and
a) means for determining the amounts of PLGF in a sample of a subject suffering from diabetes being on intensive glycemic control, and
b) means for comparing the amounts determined by the means to a reference amount, allowing predicting the risk of an acute cardiovascular event in a subject who suffers from diabetes and is on intensive glycemic control.

The terms “kit” and “device” are defined elsewhere in this specification.

It is also contemplated that the aforementioned kit or device comprises means for determining the amount at least one further marker selected from the group consisting of a natriuretic peptide and a cardiac troponin and means for comparing the amount(s) as determined by the means to a suitable reference amount(s).

Moreover, the present invention relates to the use of PLGF for predicting the risk of an acute cardiovascular event in a subject who suffers from diabetes and is on intensive glycemic control. Finally, the present invention relates to the use of PLGF and at least one further marker selected from the group consisting of a natriuretic peptide and a cardiac troponin for predicting the risk of an acute cardiovascular event in a subject who suffers from diabetes and is on intensive glycemic control.

The following Examples shall merely illustrate the invention. They shall not be construed, whatsoever, to limit the scope of the invention.

### EXAMPLE 1

The amounts of PLGF, troponin T, and NT-proBNP were determined in serum samples of 891 patients suffering from type 1 diabetes by using the commercially available assays. Plasma levels of PLGF were determined using the commercially available Immunoassays “Quantikine” (Catalog number DPG00) from R & D Systems, USA. NT-proBNP and sensitive troponin T plasma levels were detected by the corresponding commercial Elecsys assays (Roche Diagnostics). It was analyzed whether these markers correlate with mortality of any cause and non-fatal cardiovascular events in a follow-up period of twelve years. Of the 891 patients 178 patients died within the follow-up period (109 patients thereof due to cardiovascular disease). The results showed that subjects with increased levels of PLGF are at elevated risk of suffering from a cardiovascular event, particularly an acute coronary syndrome. Also, subjects with increased amounts of troponin T and NT-proBNP are at elevated risk of suffering from an acute cardiovascular event.

The results of the study are summarized in the following table.

| | |
|---|---|
| N = 891 patient | |
| Patients per quartile: n = 223 | |
| PIGF (pg/ml) | |
| 25^{th} percentile: 10 | |
| 50^{th} percentile: 13 | |
| 75^{th} percentile: 16 | |
| 95^{th} percentile: 31 | |
| PIGF | all cause mortality (total n = 178) |
| 1. Quartil | n = 27 (~12%) |
| 2. Quartil | n = 34 (~15%) |
| 3. Quartil | n = 40 (~18%) |
| 4. Quartil | n = 77 (~35%) |

Troponin T (levels in pg/ml)

| | |
|---|---|
| 25^{th} percentile: <2 | |
| 50^{th} percentile: 5 | |
| 75^{th} percentile: 11 | |
| 95^{th} percentile: 36 | |
| Troponin T | all cause mortality (total n = 178) |
| 1. Quartil | n = 5 (~2%) |
| 2. Quartil | n = 25 (~11%) |
| 3. Quartil | n = 39 (~17%) |
| 4. Quartil | n = 109 (~49%) |

NT-proBNP (median of Quartiles in pg/ml)

| | |
|---|---|
| 25^{th} percentile: <29 | |
| 50^{th} percentile: 58 | |
| 75^{th} percentile: 150 | |
| 95^{th} percentile: 788 | |
| NT-proBNP | all cause mortality (total n = 178) |
| 1. Quartil | n = 16 (~7%) |
| 2. Quartil | n = 27 (~12%) |
| 3. Quartil | n = 33 (~15%) |
| 4. Quartil | n = 102 (~46%) |

### EXAMPLE 2

A 59-year old female patients with diabetes type 2 presents at her primary physician. The amounts of PLGF, troponin T and NT-proBNP are determined (PLGF 22 pg/ml, NT-proBNP (198 pg/ml), troponin T (21 pg/ml)). The increased amounts of theses marker indicated a cardiovascular disease. The HbA1c level is determined. Since the level is increased (8.0%) a therapy that aims to significantly decrease HbA1c is initiated (medication with thiazolidinediones and insulin). The blood sugar level is measured at short intervals. After 3 month, the HbA1c level is determined again (5.9%) and the therapy is continued. After 6 months, the patient suffers from a non-fatal acute cardiovascular event.

### EXAMPLE 3

A 57 years old female patient with known diabetes mellitus has a NT-proBNP level of 80 ng/ml, a PLGF level of 9 pg/ml and a troponin T level which is below the detection limit. The patients gets 40 I.E., insulin daily (fasting glucose: 80 mg/dl, HbA1C 5.8%). The patient has even under increased physical stress no cardiac discomfort. A cardiac stress test carried out at a cardiologist (up to 250 Watt) showed no irregularities. Within the next four years of therapy (intensive glycemic control), the patient does not suffer from a cardiac event.

What is claimed is:

1. A method for identifying susceptibility of a subject to therapy for intensive glycemic control wherein the subject suffers from diabetes mellitus, the method comprising the steps of:
determining an amount of PLGF (placental growth factor) in a sample from the subject, and
comparing the amount of PLGF determined with a reference amount of PLGF, wherein susceptibility of the subject to the therapy for intensive glycemic control is indicated when the amount of PLGF determined is lower than the reference amount of PLGF.

2. The method of claim 1, wherein the subject also suffers from, or is at risk of suffering from, coronary artery disease.

3. The method of claim 1, wherein the therapy for intensive glycemic control is one that reduces glycosylated hemoglobin (HbA1c) in the patient to a level of 6.0% of total hemoglobin or lower.

4. The method of claim 1, wherein the reference amount for PLGF is 16 pg/ml.

5. A method for identifying susceptibility of a subject to therapy for intensive glycemic control wherein the subject suffers from diabetes mellitus, the method comprising the steps of:
determining amounts of PLGF (placental growth factor), a cardiac troponin and/or a natriuretic peptide in a sample from the subject, and
comparing the amount of PLGF determined with a reference amount of PLGF, comparing the amount of the cardiac troponin determined with a reference amount of the cardiac troponin, and comparing the amount of the natriuretic peptide determined with a reference amount of the natriuretic peptide, wherein susceptibility of the subject to the therapy for intensive glycemic control is indicated when the amount of PLGF determined is lower than the reference amount of PLGF, when the amount of cardiac troponin determined is lower than the reference amount of cardiac troponin, and when the amount of natriuretic peptide determined is lower than the reference amount of natriuretic peptide.

6. The method of claim 5, wherein the cardiac troponin is troponin T, the reference amount for PLGF is 16 pg/ml, and the reference amount for troponin T is 10 pg/ml.

7. The method of claim 5, wherein the natriuretic peptide is NT-proBNP, the reference amount for PLGF is 16 pg/ml, and the reference amount for NT-proBNP is 150 pg/ml.

8. A method for identifying susceptibility of a subject to therapy for moderate glycemic control wherein the subject suffers from diabetes mellitus, the method comprising the steps of:
determining an amount of PLGF (placental growth factor) in a sample from the subject, and
comparing the amount of PLGF determined with a reference amount of PLGF, wherein susceptibility of the subject to the therapy for moderate glycemic control is indicated when the amount of PLGF determined is larger than the reference amount of PLGF.

9. The method of claim 8, wherein the reference amount for PLGF is 16 pg/ml.

10. A method for predicting a risk of an acute cardiovascular event for a subject wherein the subject suffers from diabetes is on intensive glycemic control, the method comprising the steps of:
determining an amount of PLGF (placental growth factor) in a sample from the subject, and
comparing the amount of PLGF determined with a reference amount of PLGF, wherein an elevated risk of an acute cardiovascular event is predicted when the amount of PLGF determined is larger than the reference amount of PLGF.

11. The method of claim 10, wherein the reference amount for PLGF is 16 pg/ml.

12. A kit adapted for identifying susceptibility of a subject to therapy for intensive glycemic control wherein the subject suffers from diabetes mellitus according to the method of claim 5, the kit comprising:
instructions for carrying out the method,
means for determining amounts of PLGF (placental growth factor), a cardiac troponin and/or a natriuretic peptide in a sample from the subject, and
means for comparing the amount of PLGF determined with a reference amount of PLGF, comparing the amount of the cardiac troponin determined, with a reference amount of the cardiac troponin, and comparing the amount of the natriuretic peptide determined with a reference amount of the natriuretic peptide.

13. A device for identifying susceptibility of a subject to therapy for intensive glycemic control wherein the subject suffers from diabetes mellitus according to the method of claim 5, the device comprising:
means for determining amounts of PLGF (placental growth factor), a cardiac troponin and/or a natriuretic peptide in a sample from the subject, and
means for comparing the amount of PLGF determined with a reference amount of PLGF, comparing the amount of the cardiac troponin determined with a reference amount of the cardiac troponin, and comparing the amount of the natriuretic peptide determined with a reference amount of the natriuretic peptide.
